(19) United States
(12) Patent Application Publication
(10) Pub. No.: US 2023/0338453 A1
(43) Pub. Date: Oct. 26, 2023

(54) PROCESS FOR PRODUCING A HYDROLYSATE OF SCUTELLARIA ROOT, WHICH IS ENRICHED AND STANDARDIZED WITH FLAVONOIDS OF INTEREST, THE HYDROLYSATE OBTAINED BY SUCH A PROCESS, AND COSMETIC AND DERMOCOSMETIC APPLICATIONS OF SUCH A HYDROLYSATE

(71) Applicant: EXSYMOL, Monaco (MC)

(72) Inventor: Pierre E. BONDON, Monaco (MC)

(21) Appl. No.: 17/908,481

(22) PCT Filed: Mar. 4, 2021
(86) PCT No.: PCT/EP2021/055482

§ 371 Date: Aug. 31, 2022

(30) Foreign Application Priority Data
Mar. 4, 2020 (FR) 2002185

(51) Int. Cl. A61K 36/539; A61K 31/352; A61K 8/9789; A61Q 19/08

(52) U.S. Cl. CPC A61K 36/539; A61K 31/352; A61K 8/9789; A61Q 19/08; A61K 2236/333; A61K 2800/805

(57) ABSTRACT

The invention relates to a process for producing a hydrolysate of Scutellaria roots enriched and standardized in flavonoids of interest. The invention also relates to the hydrolysate resulting from such a process, a composition comprising such a hydrolysate, as well as cosmetic and dermocosmetic applications of such a hydrolysate.

### FIELD OF THE INVENTION

The present invention relates to a process for obtaining a hydrolysate obtained from a plant material, enriched and standardized in metabolites of interest, the hydrolysates resulting from such a process and the compositions comprising such hydrolysates, as well as their cosmetic and dermocosmetic applications.

### STATE OF THE ART

Thousands of years old, Traditional Chinese Medicine (acronyms “TCM”) is one of the oldest medicines in the world used to maintain or restore health. It brings together all the knowledge, know-how and health practices belonging to the history of Chinese civilization. It has multiple tools to help the body to stay in good health or to recover it, mainly pharmacopoeia, acupuncture, massage, energy exercises and dietetics. However, the first preferred approach remains the traditional pharmacopoeia where medicinal plants occupy a prominent place due to their high value attributed in Chinese culture and traditions. Thus in China and with several thousand plants listed in national herbalism, the use, both preventive and curative, of herbal preparations is still very much practiced today alongside modern medicine.

Among the most popular preparations and plant species in traditional Chinese medicine is, for its root part, a perennial flowering plant of the Lamiaceae family, Lake Baikal skullcap (Latin botanical name: Scutellaria baicalensis Georgi). Indeed, once dried, the underground organ of this plant has been the basis, for more than 2,000 years, of a traditional remedy highly prized in China called “Huang-Qin” (pharmaceutical name: radix Scutellariae), intended to prevent or combat disorders and pathological conditions of the body as heterogeneous as diarrhoea, dysentery, hypertension, haemorrhage, insomnia, respiratory infections, influenza, pneumonia and cancer (Zhao Q. et al., Sci. Bull., 2016, vol. 61, pp. 1391-1398 and references cited). Proof of its recognition by the contemporary medical community, “Huang-Qin” is now registered in the European and British pharmacopoeias, in addition to its more distant registration in the Chinese pharmacopoeia (Wang Z.L. et al., Pharm. Biol., 2018, vol. 56, pp. 465-484). In France, Lake Baikal skullcap is also included in the decree of Jun. 24, 2014 which establishes the list of plants, other than mushrooms, authorized in food supplements. Taxonomically, the Lake Baikal skullcap belongs to the botanical genus “Scutellaria” which includes around the planet about 350 species, sometimes more commonly referred to as “skullcaps” (Shang X. et al., J. Ethnopharmacol., 2010, vol. 128, pp. 279-313 and references cited). Its distribution is not limited to the surroundings of the Siberian lake and more widely to the Russian Federation. The Scutellaria baicalensis species is also found as a native plant in several East Asian countries: China, Mongolia, Japan, Korean Peninsula (Shang X. et al., J. Ethnopharmacol., 2010, vol. 128, pp. 279- 313). Due to a growing demand for such a plant resource and to avoid its overexploitation, it is also cultivated on a large scale in several Chinese provinces, in Europe and particularly in Poland (Kosakowska 0., Herba Pol., 2017, vol. 63 , pp. 20-31), or even in the state of Mississippi in the United States (Zheljazkov V. D., HortScience, 2007, vol. 42, pp. 1183-1187).

The literature reveals that the root and aerial parts of Scutellaria baicalensis constitute the two organs of the plant for which pharmacognosy work has been carried out in order to study their medicinal potential. The analysis of scientific publications reveals, however, that it is the root of the plant which arouses, by far, the most interest (Chirikova N. K. et al., Russian J. Bioorg. Chem., 2010, vol. 36, p. 909-914 and references cited). It is true that Scutellaria baicalensis displays, among the hundreds of molecules identified in its root parts, a wide spectrum of pharmacologically renowned bioactive metabolites, and in particular more than 80 flavonoids accumulated and stored in free or glycoconjugated form (Wang Z. L. et al. ., Pharm. Biol., 2018, vol. 56, pp. 465-484), for a total content that can reach or exceed 20-25% of dry matter (Blach-Olszewska Z. B. et al., Adv. Clin. Exp. Med., 2008, vol. 17, pp. 337-345). Thus with such a phytochemical profile, it is not surprising to see attributed to aqueous or alcoholic extracts from the roots of Scutellaria baicalensis and titrated in certain flavonic derivatives, a wide range of activities on the organism: anti-tumor, antiviral, antimicrobial, anti-inflammatory, antioxidant, neuro-protective, hepato-protective, anticonvulsant, etc. (Wang Z. L. et al., Pharm. Biol., 2018, vol. 56, pp. 465-484 and references cited).

This same literature around the qualitative and quantitative evaluation of the metabolites present in the roots of the plant agrees on the fact that the flavonoids designated “baicalin” (5,6-dihydroxyflavone-7O-β-D-glucuronide in systematic chemical nomenclature), “wogonoside” (5,7-dihydroxy-8-methoxyflavone-7-O-β-D-glucuronide), combined with their corresponding aglycone derivatives “baicalein” (5,6,7-trihydroxyflavone) and “wogonin” (5,7-dihydroxy-8-methoxyflavone), constitute, in combination, the most abundant metabolites among those identified in the roots of the plant (Li C. et al., Biopharm. & Drug Disposition, 2011, vol. 32, pp. 424-445; Zhao Q. et al., Sci. Bull., 2016, vol. 61, pp. 1391-1398). It is also reported for this plant that the free forms (aglycones) of these preeminent metabolites are pharmacologically more active than their glycosylated analogues, thus suggesting the essential role played by such aglycone flavonoids in the clinical therapeutic efficacy of “Huang- Qin” mentioned above (Wang Z. L. et al., Pharm. Biol., 2018, vol. 56, pp. 465-484 and references cited).

With regard to the aglycones baicalein and wogonin, such a distribution in the plant kingdom has aroused great interest due to the numerous virtues on the organism that they individually exhibit. Baicalein is thus recognized for its cytotoxic power against metastases and several cancers (Liu H. et al., Int. J. Mol., 2016, vol. 17, pp. 1-18). Wogonin also appears among the effective anticancer strategies, due to potent antitumor and apoptotic activities (Wu X. et al., Onco Targets and Ther., 2016, vol. 6, pp. 2935-2943). In skin care, wogonin is found to be effective in the treatment of skin hyperpigmentation, as a bleaching agent (Kudo M. et al., PLos One, 2017, vol. 12, pp. 1-26). Recently studied on epidermal keratinocytes in the context of dermatosis, baicalein demonstrated an ability to inhibit the production of pro-inflammatory interleukins (Da X. et al., J. Dermatol. Sci., 2019, vol. 95, pp. 84-87). Finally, wogonin and baicalein both prove to be advantageous in the correction of skin damage induced by prolonged exposure to ultraviolet radiation (Kimura Y. et al., 2011, vol. 661, pp. 124-132).

### SUMMARY OF THE INVENTION

Also, based on all these findings, in response to a growing demand for the integration of active ingredients of natural origin that are innovative, safe and effective in cosmetic products, the applicant became interested in the design of a new extract of Scutellaria baicalensis, on the one hand with a content enriched in wogonin and baicalein after a controlled conversion of their glycoside precursors, on the other hand defined by a normalized ratio between these two aglycone target markers.

By “normalized ratio”, it is necessary to understand a mass ratio (weight/weight) wogonine to baicaléine established after a comparative screening of several of their synthetic replicas in mixture, aiming to define a ratio between these two markers likely to confer on the new extract a biological behavior on the skin that can be the most favorable possible. At the origin of such a determination, it is indeed noticed in the qualitative evaluation carried out on batches of skullcap from various horizons and intended for specific medicinal preparations, the attention paid, no longer to the sole quantification of phytochemical mono-constituents, but now also to ratios between prominent flavonoids (Shang X. et al., J. Ethnopharmacol., 2010, vol. 128, pp. 279-313).

By “controlled conversion”, it is necessary to understand the identification of operating conditions which make it possible to simultaneously maintain solubilization and stabilization of the released aglycone flavonoids of interest.

As a limiting factor for such controlled conversion, it is known that flavonoids in general are chemically unstable (Wang J. et al., J. Serb. Chem. Soc., 2016, vol. 81, pp. 243-253), such instability being liable to cause their degradation and in particular, for example, the occurrence of unfavorable auto-oxidation or dimerization reactions. Another characteristic pitfall of aglycone flavonoids in general is their virtual insolubility in the usual solvents and their mixtures, other than short-chain alcohols, which leads them in particular, in an essentially aqueous hydrolytic medium and as and when they form, to precipitate or to crystallize. Yet frequently used in obtaining plant extracts, a traditional extraction of the hydro-alcoholic type, in particular using an ethanol/water mixture, should be discarded in the face of the uncertainty that all of the metabolites originally present in the plant has been extracted and therefore only part of the aglycone forms are present in the final product. In addition, this type of extraction requires often flammable short-chain alcoholic solvents, such as ethanol, which therefore become incompatible with an industrial scale.

Consequently, and taking into account the foregoing, the technical problem which the present invention proposes to solve is to develop, on an industrial scale, a process for obtaining a hydrolyzed extract of roots of Scutellaria baicalensis enriched and standardized in flavonoids of interest under their free form (aglycone). More specifically, said problem consisted in researching and identifying extraction, hydrolysis and processing conditions that allow maximum and non-denaturing conversion of the glycosides baicalin and wogonoside into their respective aglycones baicalein and wogonine, coupled with optimal recovery in solution of said aglycones, without any purification step. To such an enrichment problem was inserted the quest for a wogonin to baicalein mass ratio close to a minimum value of about 0.2 after the applicant had established it prior to the design of the hydrolyzed extract during an experimental test aimed at identifying the wogonin to baicalein ratio carrying the most favorable score as an agent capable of limiting the secretion of a mediator of cutaneous inflammation [cf. test 1 below].

### DESCRIPTION OF THE INVENTION

To achieve these objectives and in particular that already of a maximum conversion of the glycoconjugates, the Applicant has naturally turned to enzymatic hydrolysis techniques. The exploitation of endogenous hydrolases appeared to them in fact as an opportunity to solve the technical problem posed by the invention, especially since their presence is reported in large numbers in this Baikal skullcap, in particular β-D glucuronidase and β-D-glucosidase, and although the presence of other hydrolases is uncertain, united as β-D-glycosidases (Yu C. et al., Pharm. Biol., 2013, vol. 51, pp. 1228-1235 and cited references). On this basis, the Applicant took advantage, initially, by an activation step, of this endogenous activity of the plant. Then in a second step, and this is the basis of the present invention, the Applicant discovered that a sequential procedure for extraction, hydrolysis and recovery of the metabolites of interest carried out according to an appropriate panel of operating conditions, allowed, without compromising a denaturation of the hydrolysis products, to access concomitantly a complete release in solution of the two aglycone forms of interest in accordance with the pre-defined normalized ratio. In particular, it has been observed that, contrary to technical prejudice, the introduction of a certain type of organic solvent into the hydrolysis medium, in this case a dihydric alcohol and preferentially 1,3-propanediol, in compliance with strict operating conditions and in particular pH, temperature and water/miscible solvent ratio, did not ultimately affect the endogenous enzymatic hydrolysis, but on the other hand behaved as an appropriate solubilizing and stabilizing agent of hydrolysis products of interest. Therefore, the development of such a process has been a satisfactory response to the objective stated above, to which are added the following other advantageous characteristics:
homogenization of the reaction medium with the addition of such dihydric alcohol without compromising or disturbing the kinetics of hydrolysis of the flavonoid glycosides, nor a fortiori causing denaturation of the endogenous enzymes of the plant as the state of the art however foreshadowed with the well-known denaturation of proteins and enzymes in the presence of alcohols (Booth N., Biochem. J. 1930, vol. 24, pp. 1699-1705) or water/organic co-solvent mixtures (Mozhaev V. V. et al., Eur. J. Biochem., 1989, vol. 184, pp. 597-602). Thus with such optimal solubilization and stabilization of the two aglycone flavonoids of interest in such a physiologically acceptable solvent for cosmetic use, it is possible to ultimately obtain a hydrolyzed extract according to a simple, rapid process, easily applicable on an industrial scale, and qualified as “one-pot”, that is to say having led to an extract that can be used directly in cosmetics;
obtaining a hydrolyzed extract with stability and physico-chemical and organoleptic characteristics (color, clarity) preserved over the long term [cf. test 2 below], also intrinsically endowed with bactericidal properties, particularly with the use of 1,3-propanediol, thus avoiding the addition of antibacterial additives;
finally, and this is another important aspect of the invention, obtaining a hydrolyzed extract with a biological behaviour very much superior to that of a native extract vis-à-vis a matrix metalloprotease involved in the proteolysis of the extracellular matrix of the skin [cf. test 3 below]. By “native extract”, we mean an extract not having undergone the hydrolytic action of the endogenous enzymes of the plant after their activation, consequently an extract which is not hydrolyzed and which has a largely majority content of flavonoid glycosides.

Concerning a state of the art likely to come close to the solution to the problem posed by the invention, it should be noted an article having as its object, for exclusively analytical purposes, a kinetic study carried out on the species Scutellaria baicalensis aimed at observing, in a context of “pre-treatment” of the plant intervening in the preamble to a subsequent extraction step, the incidence of major physico-chemical parameters capable of influencing the conversion of flavonoid glycosides into their corresponding aglycone derivatives (Yu C. et al., Pharm. Biol., 2013, vol. 51, pp. 1228-1235 and cited references). However, contrary to the present invention, it is not proposed in any way a technical solution for their optimal recovery in solution in the form of an extract hydrolyzed and normalized in metabolites of interest. Processs aimed at biocatalyzing the conversion of the glycosides wogonoside and/or baicalin into their respective aglycones are also noted in the prior art, but with the exogenous addition of glycosidases (Yu C. et al., Oncology Reports, 2013, vol. 30, pp. 2411-2418) or even a particular fermenting microorganism (Yun M. Y et al., Pharmacogn. Mag., 2018, vol. 14, S453-7). Finally, in terms of extraction solvent, ethanol is announced to be the optimal solvent for the design of extracts of Scutellaria baicalensis Georgi for biopharmaceutical purposes in topical application (Vytis Cizinauskas, thesis extract, May 2013). Ethanol is also used in a recent study aimed at observing the hydrolysis kinetics of baicalin during extractions carried out on roots of Scutellaria baicalensis (Boyko. N. N. et al., Pharm. & Pharmacol., 2019, vol. 7, pp. 129-137).

Consequently, the present invention therefore has as its first object a process for the production of a hydrolysate of roots of the genus Scutellaria enriched and standardized in wogonin and baicalein, comprising the following steps:
(i) a phase for activating endogenous enzymes, preferably one or more β-glycosidases, by dispersion, with mechanical stirring and for a period of between about 10 minutes and about 1 hour, of said roots, in a root/water weight ratio from about 0.1 to about 0.5;
(ii) a phase of enzymatic hydrolysis of the dispersion obtained in step (i) by thermochemical means, said phase comprising the following steps:
   (a) the addition of a miscible organic solvent of the dihydric alcohol type in a solvent/water weight ratio of about 1 to about 5;
   (b) heat treatment of the reaction medium obtained in step (ii/a) at a temperature of between about 40 and about 55° C., and for a period of between about 20 hours and about 50 hours;
(iii) a phase of irreversible inhibition of the enzymatic activity in the reaction medium obtained in step (ii) by heat treatment at a temperature of between about 65° C. and about 90° C. and for a period of between about 1 hour and about 3 hours;
(iv) a phase of adjusting the pH to a value between about 3.5 and about 5.5;
(v) a phase of adjustment of the mass of crude hydrolysate by adding a solvent of the dihydric alcohol type in order to guarantee a hydrolysate reduced to its dry extract at a variable content of about 1.8 to about 2 .5%.

By “hydrolysate of roots of the genus Scutellaria enriched and standardized in wogonin and baicalein” as obtained at the end of step (v), we mean above all, in the first place, a hydrolysate which fulfills at least one of the criteria below:
a hydrolysate enriched and standardized in wogonin and baicalein in free form (aglycone), in particular by comparison with a traditional extract of hydro-alcoholic type carried out for the usual analysis of the phytochemical constituents of a plant in its native state;
a hydrolysate enriched and standardized in wogonin and baicalein and in which the wogonin/baicalein weight ratio is at least about 0.2;
a hydrolysate comprising a wogonin molar content of between about 1 and about 5 mM (i.e. a weight content of between about 0.28 and about 1.40 mg/g);
a hydrolysate comprising a baicalein molar content of between about 10 and about 15 mM (i.e. a weight content of between about 2.7 and about 4.05 mg/g);
a hydrolysate comprising a weight content of wogonoside of less than about 50 ppm (i.e. less than about 0.05 mg/g);
a hydrolysate comprising a weight content of baicalin of less than about 50 ppm (i.e. less than about 0.05 mg/g);
a hydrolysate having a conversion rate of wogonoside to wogonin of at least about 95%;
a hydrolysate having a conversion rate of baicalin to baicalein of at least about 95%;
a hydrolysate having a solubilized wogonin recovery rate of at least about 80%;
a hydrolysate having a solubilized baicalein recovery rate of at least about 60%.

Secondly, the term “hydrolysate of roots of the genus Scutellaria enriched and standardized in wogonin and baicalein” should be understood as a hydrolysate which fulfills at least one of the following criteria:
a hydrolysate enriched and standardized in wogonin and baicalein and in which the wogonin/baicalein weight ratio is at most about 0.4;
a hydrolysate that has not undergone any stage of separation by chromatography;
a hydrolysate directly usable in cosmetics, in particular in the context of a cosmetic formulation.

According to a preferred embodiment of the invention, the term “roots of the genus Scutellaria” according to the invention means roots previously ground or pulverized, and optionally reduced to the state of powder. These roots come from one or more of the species Scutellaria baicalensis, Scutellaria amoena, Scutellaria barbata, Scutellaria hypericifolia, Scutellaria lateriflora, Scutellaria racemosa, Scutellaria tomentosa, Scutellaria viscidula, Scutellaria wrightii and Scutellaria planipes with regard to the announced similarity of their phytoconstituents (Chiang Chan E. W. et al., J. Chin. Pharma. Sci., 2019, vol. 28, pp. 217-228; Shang X. et al., J. Ethnopharmacol., 2010, vol. 128, pp. 279-313). Advantageously, these are roots from the species Scutellaria baicalensis or Scutellaria lateriflora. In particular, these are roots from the species Scutellaria baicalensis.

According to a preferred embodiment of the invention, the roots are crushed or ground, and reduced to a powder state.

According to a preferred embodiment of the invention, the root/water weight ratio of step (i) is about 0.2.

According to a preferred embodiment of the invention, the solvent/water weight ratio of step (ii/a) is about 4.

According to a preferred embodiment of the invention, the miscible organic solvent of dihydric alcohol type of step (ii/a) is advantageously chosen from 1,3-propanediol, 1,2-propanediol, methylpropanediol, phenoxypropanediol, 1,3-butanediol, 1,2-butanediol, 1,4-butanediol, 2,3-butanediol, 1,2-hexanediol, 1,2-dihydroxyethane, 3-oxapenta-1 ,5-diol, and mixtures thereof. Advantageously, it is 1,3-propanediol, 1,2-propanediol, 1,3-butanediol or a mixture of these solvents. Most particularly, it is 1,3-propanediol.

According to a preferred embodiment of the invention, the temperature in the heat treatment of step (ii/b) is between about 45° C. and about 55° C., preferably about 50° C.

According to a preferred embodiment of the invention, the duration of the heat treatment of step (ii/b) is between about 20 hours and about 30 hours, preferably about 24 hours.

According to a preferred embodiment of the invention, the temperature in the heat treatment of step (iii) is between about 65° C. and about 75° C., preferably about 70° C.

According to a preferred embodiment of the invention, the duration of the heat treatment of step (iii) is between about 1 hour 30 minutes and about 2 hours 30 minutes, preferably about 2 hours.

According to a preferred embodiment of the invention, the adjustment pH of step (iv) is about 4.5.

According to a preferred embodiment of the invention, the content of the hydrolysate reduced to its dry extract from stage (v) is about 2%.

By way of illustrative example of the first object of the invention, a hydrolysate of roots of the genus Scutellaria enriched and standardized in aglycone flavonoids of interest is prepared according to a process comprising the following steps:
(i) activation of the endogenous β-glycosidases of 25 g of ground roots of Scutellaria baicalensis by suspending them with mechanical stirring in 100 g of water;
(ii) enzymatic hydrolysis of the dispersion with the addition of 75 g of 1,3-propanediol and reaction medium brought to a temperature of 50° C. for 24 hours;
(iii) reaction medium brought, after addition of 350 g of 1,3-propanediol, to a temperature of 70° C. for 2 hours, followed, after returning to room temperature, by filtration to obtain a clarified filtrate;
(iv) addition of a few drops of 6N HCl to the reaction medium to readjust the pH to 4.5;
(v) adjustment of the mass of crude hydrolysate by 94 g of 1,3-propanediol in order to guarantee the content of the hydrolysate reduced to its dry extract at 2%, and ultimately obtain, after stirring, 526 g of a clear hydrolysate, enriched and standardized in wogonin and baicalein, called “hydrolysate of Scutellaria roots (batch 37582)” in the formulations and the activity tests below, and containing and/or displaying under these conditions:
0.87 mg/g (i.e. 3.1 mM) of wogonin;
3.43 mg/g (i.e. 12.7 mM) of baicalein;
a wogonin to baicalein weight ratio of 0.25;
an undetectable amount of baicalin, at least less than 50 ppm (i.e. 0.05 mg/g);
an undetectable amount of wogonoside, at least less than 50 ppm (i.e. 0.05 mg/g);
an amount of solubilized wogonin with a recovery rate of 87%;
an amount of solubilized baicalein with a recovery rate of 65%;
a wogonin enrichment factor close to 6 (5.8);
a baicalein enrichment factor close to 10 (9.8).

The present invention also relates to a hydrolysate of roots of the genus Scutellaria enriched and standardized in wogonin and baicalein, obtainable by the process as defined above.

Another object according to the invention is a hydrolysate of roots of the genus Scutellaria enriched and standardized in wogonin and baicalein obtained by the process according to the invention, for its use in cosmetics or dermocosmetics since, in addition to its superior behaviour compared to a non-hydrolysed extract [cf. test 3 below], said hydrolysate also presented other advantageous responses for the skin, illustrated by the following tests:
an ability to protect dermal fibroblasts from a premature senescent state induced by a stressful situation, due to its inhibitory effect on the activity of a cutaneous hydrolase marker of such a state, SA-β-galactosidase (beta-galactosidase associated with senescence) [cf. test 4 below];
an ability to protect dermal fibroblasts from a premature senescent state induced by a stressful situation, due to its reducing power on the cell to excessively stimulate the synthesis of its endogenous glutathione to oppose said stress [cf . test 5 below];
an ability to protect dermal fibroblasts from a premature senescent state induced by a stressful situation, due to its antioxidant power on the production of reactive oxygen species [cf. test 6 below];
an ability to protect dermal fibroblasts from a premature senescent state induced by a stressful situation, due to its inhibitory effect on the secretion of exosomes (secretory phenotype associated with cellular senescence) [cf. test 7 below].

By “dermocosmetics” is meant an action that is both cosmetic and dermatological. According to a preferred embodiment of the invention and because of its antioxidant properties in particular, the cosmetic use of a hydrolysate of roots of the genus Scutellaria enriched and standardized in wogonin and baicalein and obtainable by the process according to the invention relates to the maintenance of homeostasis of the microbiome at the surface of the skin.

According to another preferred embodiment of the invention, the (dermo)cosmetic use of a hydrolysate of roots of the genus Scutellaria enriched and standardized in wogonin and baicalein and obtainable by the process according to the invention relates to the prevention or fight against the effects of visible radiation (VIS: λ400-700 nm), in particular against blue light (λ380-470 nm), in particular for its inhibiting effect on the overproduction of the matrix metalloproteinase MMP-1 induced by blue light.

Another preferred embodiment of the invention relates to the (dermo)cosmetic use of a hydrolysate of roots of the genus Scutellaria enriched and standardized in wogonin and baicalein and obtainable by the process according to the invention, in the prevention or fight against the skin signs resulting from stress such as air pollution, contact with chemical xenobiotics or smoky atmospheres.

Another preferred embodiment of the invention relates to the cosmetic use of a hydrolysate of roots of the genus Scutellaria enriched and standardized in wogonin and baicalein and obtainable by the process according to the invention, as an energizing agent of the skin. According to another preferred embodiment of the invention and because of its soothing virtues in particular, the dermocosmetic use of a hydrolysate of roots of the genus Scutellaria enriched and standardized in wogonine and baicaléine and obtainable by the process according to the invention relates to the treatment of redness and other itching on the surface of the skin.

Another preferred embodiment of the invention is the (dermo)cosmetic use of a hydrolysate of roots of the genus Scutellaria enriched and standardized in wogonin and baicalein and obtainable by the process according to the invention in the prevention or the fight against cellular senescence and in particular against the deleterious effects of the secretory phenotypes associated with it.

Another preferred embodiment of the invention is the use of the hydrolysate of roots of the genus Scutellaria enriched and standardized in wogonin and baicalein, obtainable by the process according to the invention, as an inhibitor of the metalloproteinase MMP -1, in particular in the treatment of any pathology or disorder in which this metalloproteinase is involved.

Finally, a last object of the invention relates to a composition, for cosmetic or dermocosmetic use, comprising, as main active ingredient, a hydrolysate of roots of the genus Scutellaria enriched and standardized in wogonin and baicalein as defined above, in combination with one or more adjuvants physiologically compatible with the skin or the appendages.

Advantageously, the amount of hydrolysate of roots of the genus Scutellaria enriched and standardized in wogonin and baicalein as defined above in the target compositions is between 0.05% and 5% by weight relative to the total weight of the composition, preferably between 0.5% and 2.5% by weight.

The compositions according to the invention are suitable for administration by the topical cutaneous route, presented in all the forms normally used for such administration. By way of indication and not limitation, the compositions may be in the form of suspensions, lotions, creams, aqueous or hydroalcoholic gels, powders and multiple emulsions which may optionally be microemulsions or nanoemulsions, etc.

The compositions according to the invention may contain, as physiologically acceptable adjuvant, at least one adjuvant known to those skilled in the art and acceptable in the cosmetic or dermocosmetic fields, chosen from oils, waxes, silicone elastomers, surfactants, co-surfactants, thickeners and/or gelling agents, humectants, emollients, organic or inorganic sunscreens, photostabilizers, preservatives, colorants, mattifying agents, tensing agents, sequestrants, perfumes, etc., and their mixtures.

The compositions according to the invention may also comprise one or more additional active agents, chosen, without this list being exhaustive, from deglycating agents, agents stimulating the synthesis of collagen or elastin or preventing their degradation, agents stimulating the synthesis of glycosaminoglycans or proteoglycans or preventing their degradation, agents increasing cell proliferation, pro-pigmenting agents, antioxidants or anti-free radicals or anti-pollution agents, moisturizing agents, draining or detoxifying agents, anti-inflammatory agents, desquamming agents, soothing and/or anti-irritant agents, agents acting on the microcirculation (anti-dark circles), agents reinforcing the cutaneous defense systems, agents favorable to the reinforcement of the barrier function, agents stimulating cell metabolism: cell growth, production of useful biomolecules for the skin such as collagen, agents opposing the harmful effects of psychological stress, molecular chaperones, agents limiting the disadvantages associated with an overproduction of sebum, agents targeting certain cutaneous pathologies such as atopic dermatitis, rosacea, healing disorders, and their mixtures.

Very particularly in the uses and compositions according to the invention, the hydrolysate of roots of the genus Scutellaria enriched and standardized in wogonin and baicalein obtainable by the process according to the invention is a hydrolysate of crushed roots of Scutellaria baicalensis.

The present invention is illustrated below, purely by way of indication, by the following examples of composition according to the invention containing a hydrolysate of roots of the genus Scutellaria enriched and standardized in the aforementioned metabolites.

Formula A (cream)
(weight percentages)

| | |
|---|---|
| Scutellaria baicalensis root hydrolysate | 2% |
| Hydrogenated polyisobutene | 7% |
| Isobutyl myristate | 3% |
| Cetyl Palmitate | 7% |
| Ethylene Glycol Monostearate | 5% |
| Lauratesorbitan | 2% |
| Polysorbate 20 | 2% |
| Carbomer (acrylate/acrylamide copolymer & | 0.3% |
| mineral oil) | |
| Phenoxyethanol | 0.5% |
| Water | qs 100% |

Formula B (gel)
(weight percentages)

| | |
|---|---|
| Scutellaria baicalensis root hydrolysate | 0.5% |
| Carbomer (acrylate/acrylamide copolymer & | 1.5% |
| mineral oil) | |
| Sodium Benzoate | 0.2% |
| Sorbic acid | 1% |
| 1,3-butanediol | 10% |
| Glycerin | 5% |
| Soda | 0.13% |
| Phenoxyethanol | 0.9% |
| Water | qs 100% |

The present invention is also illustrated below, purely as an indication, by the following tests mentioned above in the description of the invention (tests 1 to 7).

The first results obtained from in vivo studies carried out on humans are also able to illustrate the cutaneous use of the hydrolysate of the roots of Scutellaria baicalensis enriched and standardized in wogonin and baicalein according to the invention.

### Test 1: Screening and Determination of a Weight Ratio of Wogonin to Baicalein as an Agent Capable of Limiting the Secretion of the Pro-Inflammatory Cytokine type IL-6

Experimentally, the test was carried out on primary human fibroblasts (abbreviation “NHDF” for “Normal Human Dermal Fibroblasts”) isolated from breast plastic surgery from a 17-year-old female patient. These NHDFs were cultured in a complete “DMEM” culture medium (with 4.5 g/l of glucose and 10% of fetal calf serum (FCS)), then maintained in an atmosphere at 37° C. and 10% of CO₂. On D-2, the fibroblasts were seeded in 6-well plates at a rate of 1.10⁴ cells/cm². On D0, the fibroblasts were subjected to stress inducing premature senescence (150 mM hydrogen peroxide, H₂O₂, for 2 hours), then incubated at 37° C. and 10% CO₂ for 72 hours in order to leave senescence settle. On D+3, these senescent fibroblasts induced by such stress (abbreviation “SIPS-fibro” for “Stress-Induced Premature Senescent-fibroblasts”) were incubated for 24 hours in the presence of different weight ratios of wogonin to baicalein (abbreviation “W/B”) of synthetic origin. On D+4, the culture supernatants were harvested in order to perform an ELISA assay for the pro-inflammatory cytokine IL-6 (“R&DSystem kit, ref. D6050”). The results obtained were then normalized with respect to the quantity of cells per condition and subjected to statistical analysis in triplicate. The results are collated in Table 1 below, in particular expressed as % inhibition of IL-6 secretion relative to the senescent control.

TABLE 1

| | Amount of IL-6 | % inhibition |
|---|---|---|
| | secreted | of IL-6 |
| Compound | (ng/10⁶ cells) | secretion |
| Control (non-senescent NHDF) | 7.7 | N/A |
| Control (senescent NHDF/“SIPS-fibro”) | 10.1 | N/A |
| Control (senescent NHDF) + | 9.1 | −10% |
| wogonine | | |
| Control (senescent NHDF) + | 7.5 | −26% |
| wogonine/baicaleine W/B = 1/1 | | |
| Control (senescent NHDF) + | 6.9 | −32% |
| wogonine/baicaleine W/B = 0.4 (1/2.5) | | |
| Control (senescent NHDF) + | 6.2 | −39% |
| wogonine/baicaleine W/B = 0.2 (1/5) | | |
| Control (senescent NHDF) + | 7.4 | −26% |
| wogonine/baicaleine W/B = 0.1 (1/10) | | |

The results highlight that the wogonin to baicalein ratios of 0.2 and 0.4, primarily the ratio of 0.2, are the two most effective weight ratios for limiting IL-6 secretion in fibroblasts at the senescent state induced by hydrogen peroxide stress.

### Test 2: Study of Stability Under Accelerated Aging Conditions of a Hydrolysate of Scutellaria Baicalensis According to the Invention and Observation of its Physicochemical and Organoleptic Characteristics Over a Period of 28 Days

Experimentally, 2 times 5 g of a Scutellaria Baicalensis root hydrolysate according to the invention at 2% dry matter and stabilized at a pH of 4.5 (batch 37582) were placed in two glass tubes equipped with jointer plugs,. These tubes were then placed in an oven at +43° C. for 28 days. The following parameters were periodically monitored: appearance, pH, and HPLC assay of wogonin and baicalein. The results are collated in Table 2 below.

TABLE 2

| | D₀ | | | | |
|---|---|---|---|---|---|
| | (batch 37582) | D₇ | D₁₄ | D₂₁ | D₂₈ |
| Aspect | clear | — | clear | — | clear |
| | brown | | brown | | brown |
| pH | 4.5 | — | 4.5 | — | 4.5 |
| HPLC | 0.87 mg/g | — | 0.87 mg/g | — | 0.87 mg/g |
| wogonine | (3.1 mM ± 0.1) | | (3.1 mM ± 0.1) | | (3.1 mM ± 0.1) |
| assay | | | | | |
| HPLC | 3.4 mg/g | — | 3.4 mg/g | — | 3.4 mg/g |
| baicaleine | (12.7 mM ± 0.3) | | (12.5 mM ± 0.3) | | (12.5 mM ± 0.3) |
| assay | | | | | |

At the end of the 28 days of study (D28), the results in Table 2 underline that the physico-chemical and organoleptic characteristics of the skullcap hydrolysate according to the invention are not affected, with an invariant appearance and color and constant levels of wogonin and baicalein.

### Test 3: Determination and Comparison of the Capacity of a Hydrolysate of Scutellaria Baicalensis According to the Invention and That of a Non-Hydrolysed Extract of Scutellaria Baicalensis to Limit the Overexpression of the Metalloproteinase MMP-1 Involved in the Proteolysis of the Extracellular Matrix of the Skin

Experimentally, the test was carried out under the same conditions as test 1 above, with the exception of an ELISA assay carried out in the present case with the metalloproteinase MMP-1 (“Abcam kit, ref. ab100603”). In an identical manner, the results obtained were then normalized with respect to the quantity of cells per condition and subjected to statistical analysis in triplicate. The results are collated in Table 3 below, in particular expressed as % inhibition of MMP-1 secretion relative to the senescent control. They allow a comparison, for the same concentration, of the biological behaviour between a skullcap root hydrolysate according to the invention (batch 37582) and a “native extract” as defined above, that is to say a non-hydrolysed extract with the following contents in its composition (batch 37377): 1228 ppm of wogonoside (i.e. 1.22 mg/g), 153 ppm of wogonin (i.e. 0.15 mg/g), 5120 ppm of baicalin (i.e. 5.12 mg/g) and 358 ppm of baicalein (i.e. 0.35 mg/g).

TABLE 3

| | Amount of | % |
|---|---|---|
| | MMP-1 | inhibition |
| | secreted | of MMP-1 |
| Compound | (ng/10⁶ cells) | secretion |
| Control (non-senescent NHDF) | 23.3 | N/A |
| Control (senescent NHDF/“SIPS-fibro”) | 179.6 | N/A |
| Control (senescent NHDF) + hydrolysate | 120.0 | −33% |
| of Scutellaria roots (batch 37582) at a | | |
| concentration of 0.1% | | |
| Control (senescent NHDF)) + | 198.6 | +10% |
| « native extract » of Scutellaria | | |
| (batch 37377) at a concentration of 0.1% | | |

The results in Table 3 underline a markedly superior, and even diametrically opposed, biological behaviour of the skullcap hydrolysate according to the invention in comparison with that observed for a native extract of the same plant. Indeed, while the skullcap hydrolysate according to the invention, enriched in wogonin and baicalein and standardized in these two metabolites according to a wogonin to baicalein weight ratio of 0.25, demonstrates an ability to partially inhibit the secretion of cutaneous metalloproteinase, having an unfavourable proteolytic action on the skin, the non-hydrolysed extract behaved, at the same concentration, as a slightly promoting agent of the secretion of said metalloproteinase.

### Test 4: Determination and Demonstration of the Capacity of a Scutellaria Baicalensis Hydrolysate According to the Invention to Limit the Activity of SA-β Galactosidase

Experimentally, the test was carried out on primary human fibroblasts (abbreviation “NHDF” for “Normal Human Dermal Fibroblasts”) isolated from breast plastic surgery from a 17-year-old patient. These NHDFs were cultured in a complete “DMEM” culture medium (with 4.5 g/l of glucose and 10% FCS), then maintained in an atmosphere at 37° C. and 10% CO₂. On D-2, the fibroblasts were seeded in 6-well plates at a rate of 1.104 cells/cm². On D0, the fibroblasts were subjected to stress inducing premature senescence (150 mM hydrogen peroxide, H₂O₂, for 2 hours), then incubated at 37° C. and 10% CO₂ for 72 hours, in the presence or not of a hydrolysate of Scutellaria baicalensis according to the invention (batch 37582). On D+3, the activity of the SA-β-galactosidase enzyme was measured by flow cytometry using the C12FDG probe (“Invitrogen, ref. D2893”), and with, as a control of 100% SA-β-galactosidase activity, the senescent control. The results obtained were then subjected to a statistical analysis in triplicate, they are collated in Table 4 below.

TABLE 4

| | | % inhibition |
|---|---|---|
| | SA-β- | of SA-β- |
| | galactosidase | galactosidase |
| Compound | activity (%) | activity |
| Control (non-senescent NHDF) | 32 | N/A |
| Control (senescent NHDF/“SIPS-fibro”) | 100 | N/A |
| Control (senescent NHDF) + | 62 | −38% |
| hydrolysate of Scutellaria roots | | |
| (batch 37582) at a concentration of 0.05% | | |
| Control (senescent NHDF) + | 59 | −41% |
| hydrolysate of Scutellaria roots | | |
| (batch 37582) at a concentration of 0.075% | | |
| Control (senescent NHDF) + | 56 | −44% |
| hydrolysate of Scutellaria roots | | |
| (batch 37582) at a concentration of 0.1% | | |
| Control (senescent NHDF) + | 52 | −48% |
| hydrolysate of Scutellaria roots | | |
| (batch 37582) at a concentration of 0.25% | | |

The results in Table 4 underline a dose-dependent capacity of the skullcap hydrolysate according to the invention to reduce the activity of SA-β-galactosidase, in addition and notably (−38%) from the use of very low and uncommon concentrations of hydrolysate (0.05%).

### Test 5: Determination and Demonstration of the Capacity of a Hydrolysate of Scutellaria Baicalensis According to the Invention to Interact on the Expression of Glutathione

Experimentally, the test was carried out on NHDF primary human fibroblasts isolated from breast plastic surgery from a 17-year-old patient. These NHDFs were cultured in “DMEM” medium supplemented with 4.5 g/l of glucose and 10% of FCS. They were maintained in an atmosphere at 37° C. and 10% CO₂. On D-2, the fibroblasts were seeded in flasks of 150 cm² wells at a rate of 3000 cells/cm² for the control condition and 8000 cells/cm² for the senescent conditions. On D0, the NHDFs were subjected to a stress inducing premature senescence (150 mM of hydrogen peroxide, H₂O₂, for 2 hours), then incubated at 37° C. and 10% CO₂ for 72 hours, in the presence or not of a hydrolysate of Scutellaria baicalensis according to the invention (batch 37582). On D+3, the cells were recovered by scraping the flasks then centrifuged. The dry pellets were then sent frozen in dry ice in order to be analyzed by spectrofluorometry using a “SAFAS Xenius” type spectrophotometer. The results are collated in Table 5 below.

TABLE 5

| | Amount of | % of |
|---|---|---|
| | glutathion | glutathion |
| Compound | (U/g prot) | secreted |
| Control (non-senescent NHDF) | 83.17 | N/A |
| Control (senescent NHDF/“SIPS-fibro”) | 149.29 | N/A |
| Control (senescent NHDF) + | 114.92 | −23% |
| hydrolysate of Scutellaria roots | | |
| (batch 37582) at a concentration of 0.1% | | |

The results in Table 5 underline the ability of the skullcap hydrolysate according to the invention to reduce the impact of stress on the fibroblasts by allowing the latter to less stimulate the synthesis or regeneration of their endogenous protective glutathione.

### Test 6: Determination and Demonstration of the Capacity of a Hydrolysate of Scutellaria Baicalensis According to the Invention to Limit the Production of Cytoplasmic ROS

Experimentally, the test was carried out on NHDF primary human fibroblasts isolated from breast plastic surgery from a 17-year-old patient. These NHDFs were cultured in “DMEM” medium supplemented with 4.5 g/l of glucose and 10% of FCS. They were maintained in an atmosphere at 37° C. and 10% CO₂. On D-2, the fibroblasts were seeded in 6-well plates at a rate of 1.104 cells/cm². On D0, the NHDFs were subjected to a stress inducing premature senescence (150 mM of hydrogen peroxide, H₂O₂, for 2 hours), then incubated at 37° C. and 10% CO₂ for 72 hours in order to leave the senescence settle. On D+3, these senescent fibroblasts induced by such stress (abbreviation “SIPS-fibro” for “Stress-Induced Premature Senescent-fibroblasts”) were incubated for 24 hours in the presence or absence of a hydrolysate of Scutellaria baicalensis according to the invention (batch 37582). On D+4, the measurement of the reactive oxygen species present in the cytoplasm of the cell (“cytoplasmic ROS” below) is carried out by flow cytometry (ref. probe: CMH₂DCFDA) and using fluorochrome markers. The results are collated in Table 6 below.

TABLE 6

| | Amount of | % inhibition |
|---|---|---|
| | cytoplasmic | of the |
| | ROS | production of |
| | measured | cytoplasmic |
| Compound | (%) | ROS |
| Control (non-senescent NHDF) | 75 | N/A |
| Control (senescent NHDF/“SIPS-fibro”) | 100 | N/A |
| Control (senescent NHDF) + | 67 | −33% |
| hydrolysate of Scutellaria roots | | |
| (batch 37582) at a concentration of 0.075% | | |
| Control (senescent NHDF) + | 59 | −41% |
| hydrolysate of Scutellaria roots | | |
| (batch 37582) at a concentration of 0.1% | | |
| Control (senescent NHDF) + | 39 | −61% |
| hydrolysate of Scutellaria roots | | |
| (batch 37582) at a concentration of 0.25% | | |

The results in Table 6 underline a dose-dependent capacity of the skullcap hydrolysate according to the invention to reduce the production of intracellular oxidizing species (cytoplasmic ROS) by senescent cells.

### Test 7: Determination and Demonstration of the Capacity of a Hydrolysate of Scutellaria Baicalensis According to the Invention to Limit the Secretion of Exosomes

Experimentally, the test was carried out under the same conditions as test 6 above, with the exception of fibroblasts seeded in flasks of 150 cm² at a rate of 8000 cells/cm². On D+4, the senescent fibroblasts induced by the same stress (abbreviation “SIPS-fibro” for “Stress-Induced Premature Senescent-fibroblasts”) were incubated for 48 hours in the presence or absence of a hydrolysate of Scutellaria baicalensis according to the invention (batch 37582). On D+6, the exosomes are isolated from the supernatants and quantified using the “SBI kit, ref. EXOCET96A-1”. In an identical manner, the results obtained were then normalized with respect to the same quantity of cells. The results are collated in Table 7 below, in particular expressed as % inhibition of exosome secretion relative to the senescent control.

TABLE 7

| | Amount of | |
|---|---|---|
| | exosomes | % inhibition |
| | secreted | of exosomes |
| Compound | (%) | secretion |
| Control (non-senescent NHDF) | 100 | N/A |
| Control (senescent NHDF/“SIPS-fibro”) | 203 | N/A |
| Control (senescent NHDF) + | 111 | −45% |
| hydrolysate of Scutellaria roots | | |
| (batch 37582) at a concentration of 0.075% | | |

The results in Table 7 underline an ability of the skullcap hydrolysate according to the invention to reduce the secretion of exosome by senescent cells.

1. A process for the production of a hydrolysate of roots of the genus Scutellaria enriched and standardized in wogonin and baicalein, comprising the following steps:
(i) activating endogenous enzymes by dispersion, under mechanical stirring and for a period of between 10 minutes and 1 hour, of said roots, in a root/water weight ratio of 0.1 to 0.5;
(ii) performing an enzymatic hydrolysis of the dispersion obtained in step (i) by thermochemical means, said phase comprising the following steps:
a) adding a miscible organic solvent of the dihydric alcohol type in a solvent/water weight ratio of 1 to 5;
(b) heat-treating the reaction medium obtained in step (ii/a) at a temperature of between 40 and 55° C., and for a period of between 20 hours and 50 hours;
(iii) irreversibly inhibiting the enzymatic activity in the reaction medium obtained in step (ii) by heat treatment at a temperature of between 65° C. and 90° C. and for a period of between 1 hour and 3 hours;
(iv) adjusting the pH to a value of between 3.5 and 5.5;
(v) adjusting of the weight of crude hydrolysate by the addition of a solvent of the dihydric alcohol type to obtain a hydrolysate reduced to a dry extract with a content varying from 1.8 to 2.5%.

2. The process of claim 1, in which the roots are ground or pulverized, and optionally reduced to powder.

3. The process of claim 1, wherein the roots are from one or more of the species Scutellaria baicalensis, Scutellaria amoena, Scutellaria barbata, Scutellaria hypericifolia, Scutellaria lateriflora, Scutellaria racemosa, Scutellaria tomentosa, Scutellaria viscidula, Scutellaria wrightii and Scutellaria planipes.

4. The process of claim 1, wherein the roots are from the species Scutellaria baicalensis or Scutellaria lateriflora.

5. The process of claim 1, wherein the wogonin/baicalein weight ratio in the hydrolysate obtained in step (v) is at least 0.2.

6. The process of claim 1, wherein the wogonin/baicalein weight ratio in the hydrolysate obtained in step (v) is at most 0.4.

7. The process of claim 1, wherein the wogonin weight content in the hydrolysate obtained in step (v) is between 0.28 and 1.40 mg/g.

8. The process of claim 1, wherein the baicalein weight content in the hydrolysate obtained in step (v) is between 2.7 and 4.05 mg/g.

9. The process of claim 1, wherein the wogonoside weight content is less than 0.05 mg/g.

10. The process of claim 1, wherein the baicalin weight content is less than 0.05 mg/g.

11. The process of claim 1, wherein the root/water weight ratio of step (i) is 0.2.

12. The process of claim 1, wherein the miscible organic dihydric alcohol solvent of step (ii/a) is selected from 1,3-propanediol, 1,2-propanediol, methylpropanediol, phenoxypropanediol, 1,3-butanediol, 1,2-butanediol, 1,4-butanediol, 2,3-butanediol, 1,2-hexanediol, 1,2-dihydroxyethane, 3-oxapenta-1,5-diol, and mixtures thereof.

13. The process of claim 12, wherein said solvent is selected from the group consisting of 1,3-propanediol, 1,2-propanediol, 1,3-butanediol, and mixtures thereof.

14. The process of claim 12, wherein said solvent is 1,3-propanediol.

15-19. (canceled)

20. An hydrolysate of roots of the genus Scutellaria enriched and standardized in wogonin and baicalein, wherein said hydrolysate is obtained by the process of claim 1.

21. The hydrolysate of claim 20, which comprises a wogonin weight content of between 0.28 and 1.40 mg/g, a baicalein weight content of between 2.7 and 4.05 mg/g, and a wogonin/baicalein weight ratio of at least 0.2.

22. (canceled)

23. A cosmetic or dermocosmetic composition, which comprises, as the main active ingredient, a hydrolysate obtained by the process of claim 1, in association with one or more adjuvants physiologically compatible with the skin or the appendages.

24. The composition of claim 23, wherein the amount of said hydrolysate is between 0.05% and 5% by weight with respect to the total weight of the composition.

25. The process of claim 1, which comprises implementing at least one of the following conditions:
the heat treatment of step (ii/b) is carried out at a temperature of between 45 and 55° C.;
the heat treatment of step (ii/b) is carried out for a period of between 20 hours and 30 hours;
the heat treatment of step (iii) is carried out at a temperature of between 65 and 75° C.;
the heat treatment of step (iii) is carried out for a period of between 1 hour 30 minutes and 2 hours 30 minutes;
the pH is adjusted at 4.5 in step (iv).

26. A method of fighting cellular senescence which comprises administering to a subject in need thereof a hydrolysate obtained by the method of claim 1.

27. The method of claim 26, for fighting the deleterious effects of the secretory phenotypes associated with cellular senescence.
